(19) United States
(12) Patent Application Publication
(10) Pub. No.: US 2018/0163279 A1
(43) Pub. Date: Jun. 14, 2018

(54) ASSAY

(71) Applicant: THE UNIVERSITY COURT OF THE UNIVERSITY OF EDINBURGH, EDINBURGH MIDLOTHIAN (GB)

(72) Inventors: SARAH HOWIE, EDINBURGH MIDLOTHIAN (GB); KATE CUSCHIERI, EDINBURGH MIDLOTHIAN (GB); HEATHER CUBIE, EDINBURGH MIDLOTHIAN (GB)

(21) Appl. No.: 15/577,623

(22) PCT Filed: May 27, 2016
(86) PCT No.: PCT/GB2016/051544

§ 371 Date: Nov. 28, 2017

(30) Foreign Application Priority Data
May 29, 2015 (GB) 1509300.8

(51) Int. Cl. C12Q 1/70; C12Q 1/6886; G01N 33/574

(52) U.S. Cl. CPC C12Q 1/708; C12Q 1/6886; G01N 33/57484; C12Q 2600/158; G01N 2800/60; G01N 2333/523; G01N 2800/52; G01N 2800/36; G01N 2800/7028; G01N 2333/025

(57) ABSTRACT

The present invention relates to methods of determining the clinical significance of an HPV infection in a subject and associated methods, systems and kits. The method involves detection of biomarkers associated with clinically significant HPV infections and associated dysplasia.

The present invention relates to methods of determining the clinical significance of an HPV infection in a subject and associated methods, systems and kits.

### BACKGROUND OF THE INVENTION

Persistent infection with high risk (oncogenic) Human Papilloma Virus (hrHPV) causes >95% of cervical cancer. Infections of the cervix with hrHPV are common but most clear naturally.

Pre-cancerous disease is screened for in the UK and several other countries. Pre-cancerous disease, termed Cervical Intraepithelial Neoplasia (CIN), is formally classified by histology (CIN1-3) according to severity. The current accepted treatment threshold for women is CIN2.

CIN1 is the most common and most benign form of cervical intraepithelial neoplasia and usually resolves spontaneously within two years. CIN1 correlates to low grade squamous intraepithelial lesion (LGSIL or LSIL) under the Bethesda system. CIN2 is typically characterised by moderate dysplasia confined to the basal 2/3 of the epithelium. CIN3 is typically characterised by severe dysplasia that spans more than 2/3 of the epithelium, and may involve the full thickness. This lesion may sometimes also be referred to as cervical carcinoma in situ. CIN2/3 correlates to high grade squamous intraepithelial lesion (HGSIL or HSIL) under the Bethesda system. Cervical abnormalities are screened for by cytology and/or detection of high risk HPV (hrHPV), but both methods are imperfect for prediction of which women need treatment.

The development and application of suitable triage tests which can risk-stratify primary screen Human Papilloma Virus (HPV) infected women is arguably the key priority for cervical screening. Although reflex triage hrHPV+ tests to cytology is the operative modality for the randomized controlled trials of HPV primary screening, and is being used for current UK cervical screening pilots, data indicates that at least 40% of hrHPV+ screens will be cytology negative and some predictions are as high as 60% cytology negative. Management of these individuals is particularly challenging.

We therefore need alternative or adjunctive tests to cytology that can reliably inform appropriate management of hrHPV+ women. To this end, non-cytological adjunctive tests have been proposed, the ones with most traction and evidence being immunohistochemistry (IHC) for the p16 protein (either alone or in conjunction with ki67); limited genotyping involving delineation of HPV 16/18; and, viral or host methylation status. Taking these in turn, p16/ki67 still requires the preparation of a cytology slide on which a level of subjective interpretation is required, also, its evidence as a triage of primary screen positives is lacking as the significant majority of clinical-performance studies have been undertaken in the context of triage of low-grade disease. One such UK study by Szarewski et al (2012)[1] indicated that p16 IHC had a positive predictive value of 50.7% for CIN2+, which, while an improvement on HPV DNA based tests in this context, is still suboptimal. With respect to genotyping, Dijkstra et al. (2014)[2] performed an analysis based on the much cited and recently completed Dutch POBASCAM population based trial of primary HPV screening. The authors evaluated 10 potential triage strategies for the detection of CIN3+ at 2 years in screen (HPV) positive women. Application of 16/18 typing at baseline and at 6 to 12 months yielded a PPV of 25.6% meaning that over 70% of women positive for 16/18 would not have significant disease if referred to colposcopy. Of the considered scenario's the highest PPV was conferred by a single baseline cytology—yet this was still only 39.7% and with a negative predictive value (NPV) of 94.3 which is arguably too low to allow a woman to return to routine screening. Furthermore, the implementation of a two-tiered system predicated on genotyping where women who are 16/18 positive have a separate management pathway to women who are HPV positive (without 16/18), who themselves have a different management pathway to those who are HPV negative also generates operational and logistical complexities. Finally, the utility of HPV16/18 typing is likely to have diminishing returns in increasingly immunised populations. In Scotland where vaccinated women have been entering the screening programme since 2011, a highly significant reduction in HPV16/18 and closely related types has been observed[3]. With respect to methylation-assays, at time of preparation of this report, there is only one published study which has assessed performance in a primary screening context; De Strooper et al (2014)[4] showed that specificity of a CADM1/MAL host methylation assay for CIN2+ in HPV positive samples was 69.6%—however, this was only when performed in combination with standard cytological assessment and not when performed as a stand-alone test. In addition as the number of samples included in this study was small (n=250) CIN2+ outcomes were rare.

Consequently, the demand for an objective, evidence-based test which is also unaffected by the changing pattern of infection and disease brought about by immunisation is substantial.

EP2387616 and EP2641976 disclose screening methods for detecting cervical dysplasia likely to progress to carcinoma by measuring various microRNAs and mRNAs.

### STATEMENTS OF THE INVENTION

According to a first aspect the present invention provides a method of determining the clinical significance of an HPV infection in a subject, the method comprising:
providing a biological sample, preferably a tissue sample, from said subject;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection.

Preferably the subject is a human, preferably a woman, but it could be another mammal in which HPV infection occurs.

The method is suitably for detecting the presence or severity of dysplasia (e.g. cervical dysplasia) in a subject. The method of the invention can be carried out on any clinical sample where there is a need to determine the presence of an HPV-induced pre-cancerous or cancerous lesion or the risk that one will develop. Thus the method is suitably for determining the clinical significance of cellular neoplasia associated with HPV infection.

In particularly preferred embodiments, said sample is a clinical sample obtained from a human. However, the sample could be obtained from a non-human mammal.

The clinical sample may be obtained from body tissues or fluids, which are susceptible to contain human papillomavirus DNA. For example, said clinical sample may be obtained from urine, blood including without limitation peripheral blood or plasma, circulating HPV-containing cells in blood, stool, sputum, bronchoalveolar fluid, endotracheal aspirates; wounds, cerebrospinal fluid, lymph node, exudate and more generally any human biopsy tissue or body fluids, tissues or materials.

In a preferred embodiment the method is for determining the clinical significance of a cervical HPV infection, particularly, but not exclusively, for determining an indication of the risk that the subject has or will develop cervical cancer or a high grade (e.g. CIN2 or CIN3, or equivalent clinical grade(s)) pre-cancerous lesion (dysplasia). Accordingly, it is preferred that the biological sample is a cervical tissue sample that comprises cervical cells. Thus, in a preferred embodiment, where the method is for diagnosing HPV-induced cervical pre-cancerous or cancerous lesion(s), said clinical sample is obtained from cervical scrapings.

However, the method of the present invention can also be applied to other tissues where HPV infection arises, particularly where cancer associated with HPV infection is a concern. For example, oral, oropharyngeal, anal, vulvar, vaginal, and penile cancers are associated with HPV infection, and appropriate tissues can be used.

Accordingly, the invention can provide a method of detecting or predicting the occurrence of HPV-induced high-grade precancerous lesions and/or HPV-induced invasive cancers. Said HPV-induced high-grade precancerous lesion or HPV-induced invasive carcinoma can be a high-grade premalignant cervical lesion or invasive cervical cancer. The HPV can be an hrHPV infection.

As touched on above, cervical cancer is almost exclusively associated with HPV infection. Human papillomaviruses constitute a group of more than 100 types of viruses, as defined based upon variations in DNA sequence. The various HPVs cause a variety of cutaneous and mucosal diseases. Certain types may cause warts, or papillomas, which are benign (noncancerous) tumors. Others have been found to cause invasive carcinoma of the uterine cervix and other cancers. HPVs are broadly classified into low-risk and high-risk types, based on their ability to induce malignant changes in infected cells. Low risk HPV types such as 1, 2, 4, 6, 11, 13 and 32 are primarily associated with benign lesions or common warts while the high risk types, such as 16, 18, 31, 33, 35, 39, 45, 51, 52, 56, 58, 59, 66, and 68 are primarily associated with premalignant and malignant epithelial lesions.

The method can include detecting HPV infection in the subject, or it can be performed in tissue which has previously been screened for HPV infection. In preferred embodiments the method can include detecting hrHPV infection, or it can be performed in tissue which has previously been screened for hrHPV infection. hrHPV infection is associated with risk of development of cancer. Methods of screening for HPV infection, including hrHPV infection, are well known in the art. Known hrHPV strains include strains 16, 18, 31, 33, 35, 39, 45, 51, 52, 56, 58, 59, 66, and 68, and thus some or all of these hrHPV strains are suitably detected in the present invention.

Suitably the method is a diagnostic method for differentiating between subjects who have an HPV infection which is likely to resolve without treatment or which does not currently merit further investigation and/or treatment (e.g. patients where a watch-and-wait strategy is appropriate), from those in which a more active management is required, e.g. where prompt or immediate treatment is advisable. In particular, the method can be used to differentiate between patients likely to exhibit normal cervical tissue or CIN1 histology/low grade squamous intraepithelial lesion (LSIL) cytology, and those likely to have CIN2 or CIN3 histology/high grade squamous intraepithelial lesion (HSIL) cytology. In other words, the method provides a triage test for risk-stratification of HPV (e.g. hrHPV) infected subjects, e.g. women which have been identified through primary cervical screening.

The method can be used as part of a diagnostic method performed by a clinician to allow them to decide upon a suitable course of treatment. For example, the treatment can comprise the surgical removal or destruction of the affected cells, usually by loop excision (also known as loop electrosurgical excision procedure, LEEP, or large loop excision of the transformation zone, LLETZ) or cold knife conisation; other conventional treatment methods include cryotherapy, cautery, and laser ablation. Alternatively, the method can be carried out by a non-clinician (e.g. by the patient).

The method suitably involves identifying an alteration in expression levels of at least one biomarker selected from the group consisting of CCL2, CCL3, CCL4, CXCL1 and CXCL12.

Suitably the alteration can be relative to:
a pre-determined reference value;
a value derived from contemporaneously obtained tissue from the subject which is not infected with HPV;
expression levels (mRNA or protein) of at least one endogenous reference gene (e.g. a housekeeping gene); and/or
a value derived from previously obtained sample, e.g. in a previous screening process.

Typically the alteration is an increase in abundance of the biomarker protein itself, or in mRNA encoding the biomarker protein. Expression of all of the abovementioned biomarkers have been found to be increased in tissue samples which display histology/cytology associated with progression towards cancerous lesions.

In some embodiments the method suitably involves the step of obtaining a sample of cervical tissue from a subject. This is a conventional technique which is performed in routine screening processes.

Accordingly, in a further aspect the present invention provides a method of screening a subject for risk of developing cervical cancer, the method comprising:
obtaining a cervical tissue sample containing a plurality of cervical cells from the subject;
detecting the presence of HPV infection (preferably hrHPV infection) in the sample;
determining the abundance, in at least a portion of said cervical sample, of at least one biomarker, or the abundance of mRNA encoding for said at least one biomarker, the at least one biomarker selected from the group consisting of CCL2, CCL3, CCL4, CXCL1 and CXCL12; and
determining therefrom an indication of risk of the subject developing cervical cancer.

Methods of the present invention can comprise the step of isolating protein from cells in the sample. Methods of isolating proteins from a tissue sample are well-known in the art. For example, a suitable method of isolating protein from a sample comprising cervical cells is described in the specific examples below.

Methods of the present invention can comprise the step of isolating nucleic acids, especially mRNA, from cells in the sample. Methods of isolating mRNA from a tissue sample are well-known in the art. For example, a suitable method of isolating mRNA from a sample comprising cervical cells is described in the specific examples below.

A high risk of developing cervical cancer suitably correlates to likelihood of the subject exhibiting a high grade squamous intraepithelial lesion or CIN2/CIN3 histology. Such a result can guide a clinician to recommending the subject has prompt or immediate treatment to prevent or treat cervical cancer or undergoing further investigation. Methods of the present invention thus suitably comprises the step of selecting subjects to undergo further investigation (e.g. investigative colposcopy) and/or selecting subjects for treatment.

Further information about CCL2, CCL3, CCL4, CXCL1 and CXCL12 is given below:
CCL2 is also known as monocyte chemotactic protein 1 (MCP1) and small inducible cytokine A2. NCBI Reference Sequence (mRNA) NM_002982; NCBI Reference Sequence (protein) NP_002973.
CCL3 is also known as macrophage inflammatory protein 1α (MIP1α). NCBI Reference Sequence (mRNA) NM_002983; NCBI Reference Sequence (protein) NP_002974.
CCL4 is also known as Macrophage inflammatory protein-1β (MIP-1β). NCBI Reference Sequence (mRNA) NM_002984; NCBI Reference Sequence (protein) NP_002975.
CXCL1 is also known as GRO1 oncogene, GROα, KC, neutrophil-activating protein 3 (NAP-3) and melanoma growth stimulating activity, alpha (MSGA-α). NCBI Reference Sequence (mRNA) NM_001511; NCBI Reference Sequence (protein) NP_001502.
CXCL12 is also known as stromal cell-derived factor 1 (SDF-1). NCBI Reference Sequence (mRNA) NM_000609; NCBI Reference Sequence (protein) NP_000600.

The above sequence references relate to NCBI RefSeq Release 69, Jan. 7, 2015.

Preferably the method comprises determining the abundance of at least two of CCL2, CCL3, CCL4, CXCL1 and CXCL12. Accordingly, determining the expression level of the following combinations of biomarkers are envisaged:

CCL2 & CCL3; CCL2 & CCL4; CCL2 & CXCL1; CCL2 & CXCL12; CCL3 & CCL2; CCL3 & CCL4; CCL3 & CXCL1; CCL3 & CXCL12; CCL4 & CCL2; CCL4 & CCL3; CCL4 & CXCL1; CCL4 & CXCL12; CXCL1 & CCL2; CXCL1 & CCL3; CXCL1 & CCL4; CXCL1 & CXCL12; CXCL12 & CCL2; CXCL12 & CCL3; CXCL12 & CCL4; or CXCL12 & CXCL1.

Suitably the method comprises determining the abundance of at least three of CCL2, CCL3, CCL4, CXCL1 and CXCL12. Accordingly, determining the expression level of the following combinations of biomarkers are envisaged:

CCL2 & CCL3 & CCL4; CCL2 & CCL3 & CXCL1; CCL2 & CCL3 & CXCL12; CCL2 & CCL4 & CCL3; CCL2 & CCL4 & CXCL1; CCL2 & CCL4 & CXCL12; CCL2 & CXCL1 & CCL3; CCL2 & CXCL1 & CCL4; CCL2 & CXCL1 & CXCL12; CCL2 & CXCL12 & CCL3; CCL2 & CXCL12 & CCL4; CCL2 & CXCL12 & CXCL1; CCL3 & CCL2 & CXCL12; CCL3 & CCL2 & CCL4; CCL3 & CCL2 & CXCL1; CCL3 & CCL4 & CXCL12; CCL3 & CCL4 & CCL2; CCL3 & CCL4 & CXCL1; CCL3 & CXCL1 & CXCL12; CCL3 & CXCL1 & CCL2; CCL3 & CXCL1 & CCL4; CCL3 & CXCL12 & CXCL1; CCL3 & CXCL12 & CCL2; CCL3 & CXCL12 & CCL4; CCL4 & CCL2 & CXCL1; CCL4 & CCL2 & CXCL12; CCL4 & CCL2 & CCL3; CCL4 & CCL3 & CXCL1; CCL4 & CCL3 & CXCL12; CCL4 & CCL3 & CCL2; CCL4 & CXCL1 & CCL3; CCL4 & CXCL1 & CXCL12; CCL4 & CXCL1 & CCL2; CCL4 & CXCL12 & CCL3; CCL4 & CXCL12 & CXCL1; CCL4 & CXCL12 & CCL2; CXCL1 & CCL2 & CCL3; CXCL1 & CCL2 & CCL4; CXCL1 & CCL2 & CXCL12; CXCL1 & CCL3 & CCL2; CXCL1 & CCL3 & CCL4; CXCL1 & CCL3 & CXCL12; CXCL1 & CCL4 & CCL2; CXCL1 & CCL4 & CCL3; CXCL1 & CCL4 & CXCL12; CXCL1 & CXCL12 & CCL2; CXCL1 & CXCL12 & CCL3; CXCL1 & CXCL12 & CCL4; CXCL12 & CCL2 & CXCL1; CXCL12 & CCL2 & CCL3; CXCL12 & CCL2 & CCL4; CXCL12 & CCL3 & CXCL1; CXCL12 & CCL3 & CCL2; CXCL12 & CCL3 & CCL4; CXCL12 & CCL4 & CXCL1; CXCL12 & CCL4 & CCL2; CXCL12 & CCL4 & CCL3; CXCL12 & CXCL1 & CCL4; CXCL12 & CXCL1 & CCL2; or CXCL12 & CXCL1 & CCL3.

Suitably the method comprises determining the abundance of at least four of CCL2, CCL3, CCL4, CXCL1 and CXCL12. For brevity the possible combinations are not individually recited, but each individual combination of at least four biomarkers is hereby disclosed as forming part of the present invention.

Determining the abundance of more than one biomarker can be preferable to a single biomarker as it can allow for a more reliable or powerful test. This can occur for many reasons, e.g. because combining information about a plurality of markers reduces the risk that a single biomarker might have an altered abundance because of an unrelated cause and unduly skew the result, and because changes in a broader pattern of abundance levels can be highly informative. Accordingly, in embodiments of the present invention it can be preferred that at least two biomarkers selected from CCL2, CCL3, CCL4, CXCL1 and CXCL12.

CXCL1 is a particularly powerful biomarker for use in the present invention. It is present at comparatively high concentrations, which is useful as is simplifies determination of abundance. Furthermore, from a statistical point of view, it demonstrates a very marked and highly significant difference in abundance between normal and CIN1 samples versus CIN2 and CIN3 samples. Thus, in particularly preferred embodiments of the invention the abundance of CXCL1 or mRNA encoding CXCL1 is determined.

CCL4 and CCL3 are both is attractive biomarkers from a statistical point of view, but it is typically present in low abundance (typically less than 70 pg/200 μg total protein for CCL4, and typically less than 25 pg/200 μg for CCL 3). This means that, depending on the methodology used to measure its abundance, it may be comparatively difficult to reliably determine the abundance of CCL4. However, there are very sensitive methods of protein detection, and reverse transcription PCR techniques can be used to detect very low levels of mRNA, and therefore it is perfectly feasible to use CCL4 as a biomarker.

In a particularly preferred embodiment of the present invention, the method comprises determining the abundance of at least CCL2, CXCL1 and CXCL12, and/or the abundance of mRNA encoding CCL2, CXCL1 and CXCL12. Suitably the method comprises determining the abundance of CCL2, CCL3, CXCL1 and CXCL12, and/or the abundance of mRNA encoding CCL2, CCL3, CXCL1 and CXCL12.

In another embodiment of the present invention, the method comprises determining the abundance of at least CCL3, CCL4 and CXCL1, and/or the abundance of mRNA encoding CCL3, CCL4 and CXCL1.

It will be apparent to the skilled person that there is considerable freedom to interpret and process data obtained by the methods of the present invention, and how to interpret or act upon the results. It may be desirable, for example, to prioritise sensitivity or specificity, or to optimise positive predictive value or negative predictive value. Depending on the context in which the method is performed, the skilled person can therefore select appropriate methods of interpreting the results. For example, the results for different markers can be weighted in different ways, different threshold abundance levels can be applied, various statistical analyses can be applied, and one or more indicators of a potentially dubious test result can be determined.

For each of the biomarkers, there are provided below exemplary protein abundance values, provided as pg of biomarker protein per 200 μg of total protein, which can be taken as indicative of increased clinical risk (e.g. of development of cervical cancer) in embodiments of the present invention:
CCL2-23 pg/200 μg total protein or higher, suitably 25 pg/200 μg total protein or higher, preferably 30 pg/200 μg total protein or higher, can be taken as indicative of an increased risk.
CCL3-15 pg/200 μg total protein or higher, 17 pg/200 μg total protein or higher, 20 pg/200 μg total protein or higher, 22 pg/200 μg total protein or higher, 25 pg/200 μg total protein or higher, can be taken as indicative of an increased risk.
CCL4-55 pg/200 μg total protein or higher, 60 pg/200 μg total protein or higher, 65 pg/200 μg total protein or higher, can be taken as indicative of an increased risk.
CXCL1-275 pg/200 μg total protein or higher, 300 pg/200 μg total protein or higher, 400 pg/200 μg total protein or higher, 450 pg/200 μg total protein or higher, 500 pg/200 μg total protein or higher, can be taken as indicative of an increased risk.
CXCL12-300 pg/200 μg total protein or higher, 325 pg/200 μg total protein or higher, 350 pg/200 μg total protein or higher, or 375 pg/200 μg total protein, can be taken as indicative of an increased risk.

The person skilled in the art is free to formulate a wide range of calculations, e.g. via suitable algorithms, in order to obtain a desired result from processing data obtained from the methods of the present invention. There involves routine application of well-known statistical analysis techniques.

The method may of course include measuring the abundance of, or detecting the presence of, one or more additional biomarkers. For example, the method may involve detection of the abundance of one or more additional cytokines (e.g. chemokines). Additional biomarkers which can be included include CCL28, Chemerin, CXCL5, IL-16, CXCL11, CXCL9, CCL5, CCL17, GP130, or Transferrin Receptor.

The method can suitably comprise detecting the abundance of one or more reference biomarkers. For example, the method can comprise detecting the abundance of one or more proteins (or mRNA encoding said one or more proteins) which typically displays substantially constant abundance irrespective of the abundance of CCL2, CCL3, CCL4, CXCL1 and CXCL12 and/or disease state. For example, IL-8 has been identified as a suitable reference biomarker as its abundance tends to remain constant. Total protein level or mRNA level can also be used. A reference biomarker can be useful as a validation that the method has been performed correctly (e.g. biomarkers have been collected and isolated correctly) and for normalisation/calibration of the data obtained.

As set out above, the method can comprise determining protein and/or mRNA abundance. As is well-known in the art, detection of both protein abundance and mRNA abundance are useful indicators of biomarker expression levels, and both approaches are therefore appropriate in the present invention. Methods/systems of measuring protein abundance or mRNA abundance are well-known in the art, and many kits and arrays are available commercially to achieve this. It will be apparent to the skilled person that any suitable method method/system, commercial or otherwise, can be used.

In preferred embodiments of the present invention, where protein abundance is being measured, the method involves a quantitative immunoassay. For example, an ELISA assay (direct or indirect, competitive or non-competitive) is a well-known and convenient form of immunoassay. Additionally, there are a large number of commercial immunoassay platforms which could be used in the present invention. Other quantitative protein assays can also be used.

Suitably the method uses antibodies or antibody fragments adapted to selectively bind at least one of CCL2, CCL3, CCL4, CXCL1 and CXCL12. Methods of making suitable antibodies or antibody fragments are well-known in the art. Suitably the method uses suitable accessory moieties to perform an ELISA or other quantitative immunoassay. Such moieties can be directly linked to the antibodies/fragments or can be linked to secondary antibodies/antibody fragments. For example the kit can comprise one or more secondary antibodies, fluorophores, or enzymes which create detectable products (e.g. chromogenic enzymes or enzymes which create luminous products). Such accessory moieties are well-known in the field of immunoassays.

In embodiments of the present invention where mRNA abundance is to be measured, the method suitably involves reverse transcription of mRNA followed by nucleic acid amplification. However, it will be apparent that any method of determining mRNA abundance can be used. So-called, ‘next generation’ nucleic acid sequencing technologies are well known in the art, and provide suitable approaches for determining mRNA abundance.

Accordingly, in preferred embodiments, at least one nucleic acid amplification step is suitably performed. This at least one amplification step should allow the amplification of at least one target sequence derived from the relevant mRNA. As used herein, the term “nucleic acid amplification” refers to any known procedure for obtaining multiple copies of a target nucleic acid sequence or its complementary or fragments thereof, using sequence-specific probes, referred to as primers. Known amplification methods include, for example, Polymerase Chain Reaction (PCR), Ligase Chain Reaction (LCR), etc.

In one preferred embodiment, said at least one amplification step carried out in the method of the invention is a PCR amplification. Methods for carrying out PCR amplification are thoroughly described in the literature, for example in “PCR Primer: A laboratory Manual” Dieffenbach and Dveksler, eds. Cold Spring Harbor Laboratory Press, 1995. Suitably real-time PCR is used. Real-time PCR allows not only the detection of a target sequence in a clinical sample but also its quantification. Real-time PCR is widely used in molecular diagnostic, in particular, for medical biology, and in microbiology [ESPY et al., 2006, Clinical Microbiology Reviews, 2006, 19(1), 165-256].

As used herein, the term “primers” refers to an oligonucleotide sequence, typically of at least 10 nudeotides, for example from 10 to 50 nucleotides, for example, from 18 to 25 nucleotides, that is designed to hybridize with a complementary portion of a target region, and will function as the starting point for the polymerization of nucleotides (primer extension) at each amplification cycle. The region to be amplified can be substantially the entire cDNA of the biomarker mRNA, or a portion of it. Design of suitable primers is routine in the art, and the relevant mRNA sequences of the biomarkers used in the present invention are available on the reference databases discussed above.

The method may suitably comprise testing for the presence of non-HPV infections to eliminate or reduce the risk that alteration in biomarker abundance has been caused by a concomitant infection, e.g. a non-HPV viral, fungal or bacterial infection. Tests for common viral, fungal or bacterial infections are well-known in the art.

Any of the above methods can also comprise providing one or more reference nucleic acids or proteins to act as an internal control or reference point.

In a further aspect of the present invention there is a provided a method of treatment of a subject (e.g. a human subject suffering from a cervical HPV infection), the method comprising the steps of:
providing a suitable biological sample, preferably a tissue sample, from said subject;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection;
deciding whether treatment is required; and
if treatment is required, deciding upon an appropriate treatment and carrying out said treatment.

Further details of methods of assessing the biomarkers are set out above, as are conventional treatments for cervical HPV lesions.

In a further aspect, the present invention provides a method for monitoring the efficacy of a treatment for HPV infection or a lesion associated with an HPV infection in a subject, the method comprising:
providing a suitable biological sample, preferably a tissue sample, from said subject which is undergoing or which has undergone treatment;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection; and
based upon the result, assessing the efficacy of the treatment.

The method suitable comprises comparing the results obtained with the results obtained in an equivalent (typically identical) procedure carried our previously.

In a further aspect of the present invention there is a provided an apparatus, system or kit adapted for use in a method as set out above.

Suitably the apparatus, system or kit is adapted for performing high throughput analysis of protein or mRNA abundance.

In a further aspect of the present invention there is a provided a kit of parts for use in a method as set out above, the kit comprising:
reagents adapted for determining the abundance of at least one biomarker in a sample and/or reagents for determining the abundance of mRNA encoding said at least one biomarker, wherein the at least one biomarker is selected from the group consisting of CCL2, CCL3, CCL4, CXCL1 and CXCL12.

Suitably the kit comprises antibodies or antibody fragments adapted to selectively bind at least one of CCL2, CCL3, CCL4, CXCL1 and CXCL12. Methods of making suitable antibodies or antibody fragments are well-known in the art.

Suitable the kit can comprise suitable accessory moieties to perform an ELISA or other immunoassay, which can be directly linked to the antibodies/fragments or can be linked to secondary antibodies/antibody fragments. For example the kit can comprise one or more secondary antibodies, fluorophores, enzymes (e.g. chromogenic, luminous or fluorogenic). Such accessory moieties are well-known in the field of immunoassays.

The kit can suitably comprise one or more reference proteins, and suitable antibodies or antibody fragments adapted to selectively bind the one or more reference proteins, and appropriate accessory moieties.

The kit can suitably comprise one or more primer pairs adapted to amplify nucleic acid encoding CCL2, CCL3, CCL4, CXCL1 and/or CXCL12. The nucleic acid can be complimentary DNA obtained by reverse transcription from the relevant mRNA molecules. The kit can thus suitably comprise reverse transcriptase.

The kit can suitably comprise probes adapted for quantitative PCR of nucleic acids encoding the CCL2, CCL3, CCL4, CXCL1 and CXCL12.

The kit can suitably comprise one or more reference nucleic acids, and suitable primers for amplification thereof. Suitably the kit also comprises a probe for quantification of the one or more reference nucleic acids.

The kit can optionally, instruction for carrying out PCR amplification or an immunoassay.

Embodiments of the present invention will now be described, by way of non-limiting example, with reference to the accompanying drawings.

### BRIEF DESCRIPTION OF THE FIGURES

FIG. 1—Proteomic microarray of chemokine expression in pooled LBC samples from women with A) normal cervical morphology and no infection; B) hrHPV infection and mild disease (CIN1); C) hrHPV infection and moderate disease (CIN2); and D) hrHPV infection and severe disease (CIN3). Each chemokine that is present is represented by a pair of white dots. The dots in the top left and right and the bottom left corners are the internal reference positive controls for the assay.

FIG. 2—Multiplex data showing four chemokines which show significant difference between hrHPV+ve samples from women with no (normal=51) or low grade (CIN1, n=30) disease and those from women with moderate (CIN2, n=63) or severe (CIN3, n=62) disease; *p<0.05; **p<0.01; ***p<0.001.

FIG. 3—Fold increase in mRNA expression for CCL2 in LBC samples with no infection and no disease compared to samples with hrHPV infection and CIN3. **=p<0.01.

FIG. 4—Multiplex data showing four chemokines for HPV+ve samples from women with normal cytology compared to all other groups analysed by Kruskall Wallis test with Dunn's post-test. Data shown as mean+1-95% CI; NS=not significant; *=p<0.05; **=p<0.01; ***=p<0.001

FIG. 5—Multiplex data showing four chemokines for HPV-ve samples from women with normal cytology compared to all other groups analysed by Kruskall Wallis test with Dunn's post-test. Data shown as mean+1-95% CI; NS=not significant; *=p<0.05; **=p<0.01; ***=p<0.001

FIG. 6—Graph of quantitative PCR results for CCL2 mRNA levels. One dot=p<0.05; two dots=p<0.01.

FIG. 7—Graph of quantitative PCR results for CCL5 mRNA levels. One dot=p<0.05; two dots=p<0.01.

### SPECIFIC DESCRIPTION OF EMBODIMENTS OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Cervical cancer remains one of the most common cancer types affecting women worldwide. The biological pathway to cervical carcinoma begins with normal intraepithelial cells, and develops through low and then high grade dysplasia before malignancy occurs. Cytologists mark the passage to malignancy as progression from normal epithelial cells to atypical squamous cells of undetermined significance (ASCUS) to low grade squamous intraepithelial lesions (LSIL) and then high grade squamous intraepithelial lesions (HSIL) before carcinoma in situ and finally malignancy result. Histologists mark the progression from normal cells to various grades of cervical intraepithelial neoplasia (CIN1, 2 and 3), then to carcinoma in situ and finally malignancy. CIN1 is considered low grade dysplasia comparable to LSIL. CIN 2 and 3 are considered high grade dysplasia comparable to HSIL.

The current standard of care includes regular cytologic testing with a Papanicolau (Pap) smear to identify abnormalities as indicating dysplasia or carcinoma in patient cells. When high grade dysplasia is detected and confirmed by histological examination, the transformation zone of the patient's cervix is removed immediately by loop excision or cone biopsy. More radical procedures are required when carcinoma is detected. At the same time, however, the progression from normal to malignancy is not strict and the presence of low grade dysplasia does not necessarily indicate that the patient will progress to high grade dysplasia or malignancy. Significantly, the negative predictive value of cytologic methods (e.g., Pap smears) for detecting high grade dysplasia is poor. Thus, low grade dysplasia may be misdiagnosed as high grade, thereby subjecting the patient to unwarranted treatment and high grade dysplasia may be misdiagnosed as low grade dysplasia, thereby delaying appropriate treatment. Accordingly, there is a need for a diagnostic method that will accurately distinguish between low and high grade dysplasia.

The development and application of suitable triage tests which can risk-stratify primary screen Human Papilloma Virus (HPV) infected women is arguably the key priority for cervical screening.

The present inventors have determined that looking at host proteins involved in innate immunity at epithelial surfaces, such as the cervix, can form the basis of such a test. It appears that persistent infection alters the local environment and sets up a different tissue response from that seen where there is no disease or resolving infection. This type of response, that is by definition ineffective at clearing virus or virally infected cells, seems likely to involve production of relatively large amounts of reactive host proteins both in infected cells and in the immediately surrounding uninfected cells. Testing for these proteins in this context has the advantages of: —not being hrHPV type specific; not looking for the very small quantities of viral protein expressed in infected epithelial cells; and amenable to being developed as a plate based high-throughput assay does not require preparation, staining and examination of cells under a microscope.

### Example 1—Analysis of Biomarkers

Methodology

Ethics Statement

Ethical approval was obtained from Scotland A Research Ethics Committee (REF 12/SS/0034). All cervical samples were collected into ThinPrep-preservcyt liquid based cytology transport medium (Hologic, Crawley, UK). For this study, anonymised, curated, cervical smear samples were obtained from the Scottish National HPV archive, which holds Generic Scotland A Research Ethics Committee approval for Research Tissue banks (REC Ref 11/AL/0174) for provision of samples for HPV related research after approval from an Independent Scottish HPV Archive Steering Committee.

Samples

Aliquots (1.5 ml) from 350 LBC samples were obtained from the Scottish HPV archive (http://www.shine.mvm.ed.ac.uk/archive.shtml). Disease status defined by either cytology and/or cervical biopsy was obtained for each sample.

hrHPV Typing

All samples were analysed by the archive staff for hrHPV infection using a clinically validated PCR test from Abbot Molecular, RealTime High Risk HPV (http://www.abbottmolecular.com/products/infectious-diseases/realtime-pcr/hepatitis-high-risk-hpv-assay.html), following the manufacturer's instructions. This assay is a qualitative in vitro PCR assay that utilizes homogeneous target amplification and detection technology for the detection of hrHPV DNA in cervical cells collected in liquid cytology media. It detects 14 high risk HPV genotypes (16, 18, 31, 33, 35, 39, 45, 51, 52, 56, 58, 59, 66, 68) and partially genotypes 16, 18 from the other 12 high risk genotypes.

Extraction of Protein from LBC Samples

This method has been previously published by the present inventors [1]. Briefly, LBC samples are collected into 20 ml ThinPrep-preservcyt medium (Hologic, Crawley, UK) which contains 50% methanol. Briefly, 100 μl ice cold chloroform is added to 900l of sample in an Eppendorf tube on ice, vortexed, incubated on ice for 5 min and spun for 5 min at 10,000 rpm at 4° C. in a bench top microfuge. The aqueous top layer is discarded, 300 μl methanol is added and the tube vortexed, and spun for 10 min at 10,000 rpm at 4° C. in a bench top microfuge. The supernatant is removed and the pellet re-suspended in 100 μl Tris Buffered Saline. Protein concentration is determined by Pierce BCA assay (Thermo Scientific, UK).

### Proteomic Array

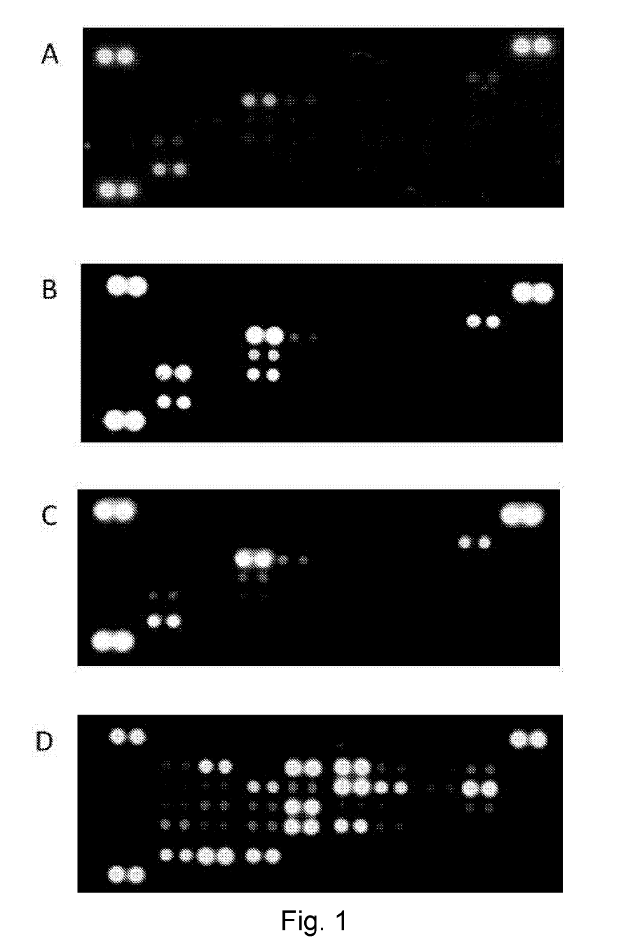

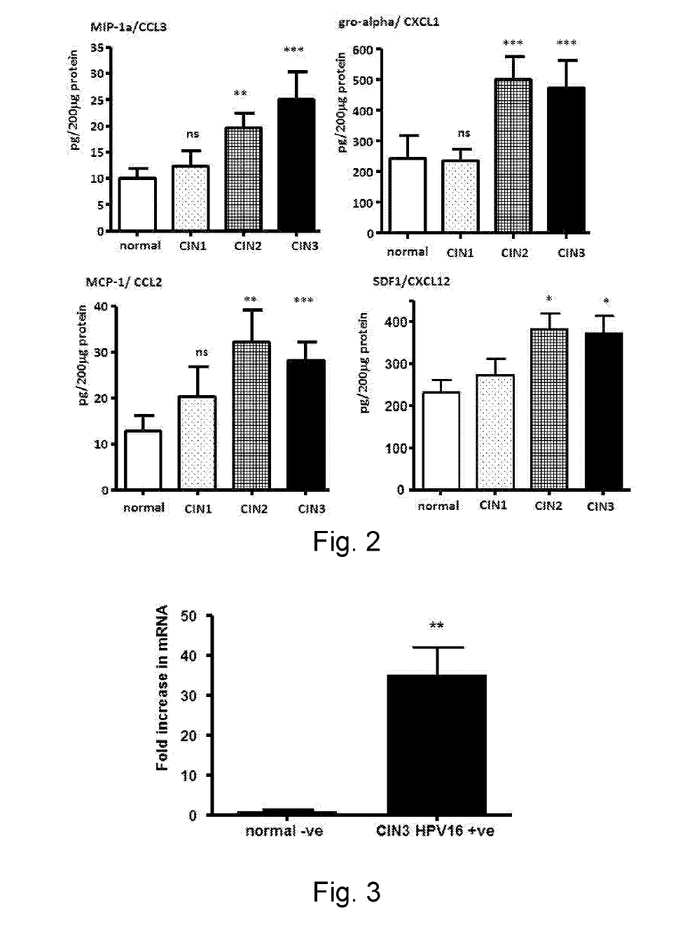

Antibody-based, human chemokine proteomic arrays (Cat No. ARY017, R&D Systems, UK) were carried out as per manufacturer's instructions. To identify proteins of interest, for the arrays, pools of samples from 9 women with different grades of disease were compared. Each pool contained 10 μg of protein from each individual sample (see Results). Developed arrays were scanned and the images reversed for use with Image-J analysis software. Each chemokine present in the pooled samples is represented by a pair of dots (see FIG. 1). Image-J software was used to measure the pixel density of each dot and the average pixel density of the two dots present for each chemokine present is given in Table 1.

### Procarta Multiplex Assay

Protein extracted as above from individual samples with different grades of disease were tested for abundance of CCL11, CCL2, CCL3, CCL4, CCL5, CXCL1, CXCL12, CXCL10 and CXCL8 by ProcartaPlex Human Chemokine Panel 1 (9-plex) supplied by Affymetrix e-Bioscience, UK, (Cat. No. EPX090-12187-901). Ten μg protein from each sample was added to a microplate well and the assay developed and read on a BioRad Bio-Plex 200 HTF multiplex assay system following the manufacturer's instructions. Data were analysed using SPSS software. Univariate analysis was done by Kruskall-Wallis rank sum test and by ANOVA; multivariate analysis was done by logistic regression.

Extraction of RNA from LBC Samples

As published[5] RNA was extracted from 1.5 ml aliquots of LBC samples using miRNeasy mini kit (Qiagen, UK) following the manufacturer's instructions. RNA was quantified on a Nanodrop spectrophotometer and stored at −80° C. until use. cDNA was made from 500 ng RNA using the Quantitect Reverse Transcription Kit (Qiagen, UK) as per manufacturer's instructions and stored at −20° C. until use. Duplex qPCR was carried out in Lightcycler Nano (Roche) real-time PCR system in duplicate in 8 well PCR strips using plates, primers, probes and reagents from Life Technologies Applied Biosystems, UK. Wells contained 2 μl cDNA, 1 μl gene of interest primer mix, 0.12 μl each of 18S forward and reverse primers, 1.6 μl 18S probe, and 13 μl mastermix. The PCR programme was 2 min at 50° C., 10 min at 95° C., followed by 40 cycles of 15 sec at 95° C. and 1 min at 60° C.

qRT-PCR for CCL2

RNA was extracted from 1.5 ml aliquots of LBC samples using miRNeasy mini kit (Cat. No. 217004, Qiagen, UK) following the manufacturer's instructions. RNA was quantified on a Nanodrop spectrophotometer and stored at −80° C. until use. cDNA was made from 500 ng RNA using the Quantitect Reverse Transcription Kit (Cat. No. 205313, Qiagen, UK) as per manufacturer's instructions and stored at −20° C. until use. Duplex qPCR was carried out in Lightcycler Nano (Roche) real-time PCR system in duplicate in 8 well PCR strips using plates, primers and probe (Cat. No. 4331182), and reagents from Life Technologies Applied Biosystems, UK. Wells contained 2 μl cDNA, 1 μl gene of interest primer mix, 0.12 μl each of 18S forward and reverse primers, 1.6 μl 18S probe, and 13 μl mastermix. The PCR programme was 2 min at 50° C., 10 min at 95° C., followed by 40 cycles of 15 sec at 95° C. and 1 min at 60° C.

### Results

Proteomic Microarray Reveals Several Chemokines are Upregulated in High Grade (CIN3) hrHPV Induced Cervical Disease

Pooled protein derived from cervical samples from women with 1) no HPV infection and no disease 2) hrHPV infection and CIN1 3) hrHPV infection and CIN2, 3) hrHPV infection and CIN3 were tested and compared for chemokine protein expression as described in Materials and Methods. FIG. 1 shows that several chemokines were up-regulated in hrHPV+ve samples from women with CIN3. The results of the analysis of pixel density shown in Table 1 demonstrate that a number of chemokine proteins are up-regulated in hrHPV+ve samples from women with CIN3 compared to uninfected controls and to hrHPV+ve infected samples from women with CIN1.

TABLE 1
Pixel density analysis of chemokine proteomic
array from pooled LBC samples.

| | Image-J calculated average pixel density | | | |
|---|---|---|---|---|
| | | HR-HPV | HR-HPV | HR-HPV |
| | NORMAL | POS | POS | POS |
| ANALYTE | HPV-ve | CIN1 | CIN2 | CIN3 |
| REF | 17856 | 20326.5 | 20302 | 18544 |
| REF | 18592.5 | 20346 | 20312.5 | 19143.5 |
| CCL21 | 0 | 0 | 0 | 938 |
| CCL28 | 0 | 0 | 0 | 17818 |
| CXCL16 | 0 | 0 | 11 | 0 |
| CHEMERIN | 0 | 0 | 0 | 18752.5 |
| CXCL5 | 0 | 0 | 0 | 18946.5 |
| CCL26 | 0 | 0 | 0 | 1371 |
| CX3CL1 | 0 | 1 | 0 | 65 |
| CXCL1 | 2039.5 | 18736.5 | 14601 | 3358 |
| CCL14 | 0 | 0 | 0 | 86.5 |
| CCL1 | 0 | 0 | 0 | 589.5 |
| CXCL8 | 11177.5 | 20351 | 20229.5 | 13834 |
| IL-16 | 996 | 1581 | 3614.5 | 4361 |
| CXCL10 | 33.5 | 0 | 0 | 18962 |
| CXCL11 | 78 | 0 | 0 | 17183 |
| LYMPHOTACTIN | 4 | 0 | 0 | 695 |
| CCL2 | 33.5 | 0 | 0 | 18878 |
| CCL7 | 0 | 0 | 0 | 420 |
| CCL22 | 223.5 | 0 | 0 | 3188.5 |
| MIDKINE | 338.5 | 12900 | 2990 | 2510 |
| CXCL9 | 100 | 4 | 0 | 19133 |
| CCL3/CCL4 | 0 | 0 | 0 | 431 |
| CCL15 | 35.5 | 0 | 0 | 167 |
| CCL20 | 0 | 0 | 0 | 0 |
| CCL19 | 48 | 0 | 0 | 1773.5 |
| CXCL7 | 1653.5 | 19882 | 1851 | 5553.5 |
| CCL18 | 0 | 9.5 | 3 | 1044.5 |
| CXCL4 | 371 | 17923 | 223 | 4083 |
| CCL5 | 82.5 | 0 | 0 | 19038.5 |
| CXCL12 | 15 | 0 | 0 | 18759 |
| CCL17 | 45 | 0 | 0 | 1007 |
| CXCL17 | 0 | 0 | 0 | 0 |
| NEG CONTROL | 0 | 0 | 0 | 0 |
| REF | 17774.5 | 20305 | 20365.5 | 18795 |

### Multiplex Chemokine Assay of Individual Samples

The threshold for treatment of women with hrHPV cervical infection is CIN2. Univariate analysis of the results showed that this platform indicated significant differences between hrHPV+ve samples from women with high grade disease (CIN2 and CIN3) and women with no or low grade (CIN1) disease for four chemokines, CCL2, CCL3, CXCL1 and CXCL12 (FIG. 1). Multivariate analysis showed that combining the data obtained on this platform for three of the chemokines CCL3, CCL4 and CXCL1 improved the sensitivity and specificity compare to any of the chemokines alone (Table 2). The most relevant data obtained in the Procarta multiplex assay is set out below in Tables 3 to 12.

TABLE 2
Multivariate logistic regression analysis of multiplex chemokine data from samples
analysed with Procarta assay as detailed in Materials and Methods.

| Analyte | AUC | Accuracy | Accuracy 95% CI | Kappa | Sensitivity | Specificity | PPV | NPV |
|---|---|---|---|---|---|---|---|---|
| CCL5 | 0.533 | 0.578 | (0.477, 0.676) | 0.138 | 0.627 | 0.512 | 0.638 | 0.5 |
| CXCL1 | 0.629 | 0.657 | (0.556, 0.748) | 0.276 | 0.78 | 0.488 | 0.676 | 0.618 |
| CXCL8 | 0.59 | 0.608 | (0.506, 0.703) | 0.196 | 0.661 | 0.535 | 0.661 | 0.535 |
| CXCL10 | 0.515 | 0.431 | (0.334, 0.533) | −0.123 | 0.407 | 0.465 | 0.511 | 0.364 |
| CCL2 | 0.638 | 0.608 | (0.506, 0.703) | 0.261 | 0.424 | 0.86 | 0.806 | 0.521 |
| CCL3 | 0.677 | 0.637 | (0.536, 0.730) | 0.302 | 0.508 | 0.814 | 0.789 | 0.547 |
| CCL4 | 0.632 | 0.618 | (0.516, 0.712) | 0.294 | 0.373 | 0.953 | 0.917 | 0.526 |
| CXCL12 | 0.594 | 0.608 | (0.506, 0.703) | 0.244 | 0.492 | 0.767 | 0.744 | 0.524 |
| All | 0.693 | 0.676 | (0.577, 0.766) | 0.347 | 0.678 | 0.674 | 0.741 | 0.604 |
| CCL3, | 0.639 | 0.608 | (0.506, 0.703) | 0.261 | 0.424 | 0.86 | 0.806 | 0.521 |
| CCL4, | | | | | | | | |
| CXCL1 | | | | | | | | |
| COMBINED | | | | | | | | |

PPV = Positive Predictive Value;
NPV = Negative Predictive Value.

mRNA Analysis by Real-Time rtPCR for CCL2 in LBC Samples Confirms Protein Data.

To reassure that the protein data was reflected at a different biological level from protein presence in the LBC samples, we extracted mRNA from aliquots of LBC samples from women with no or high grade (CIN3) disease and carried out real-time rtPCR analysis for one of the chemokines (CCL2) shown to be different at the protein level. FIG. 3 shows a significant 34-fold increase in expression of CCL2 mRNA in hrHPV+ve samples with CIN3 compared to samples from uninfected women with no disease. This indicates that mRNA can be used in place of (or in addition to) protein as a biomarker.

### Further Data—Marker Abundance as Determined by Procarta Multiplex Protein Assay

The abundance values of the chemokines CCL2, CCL3, CCL4, CXCL1 and CXCL12 correlated to disease state, as determined in the Procarta experiments, are set out below in Tables 3 to 12.

### CXCL1 (GRO-Alpha, pg Protein Per 200 μg Protein)

TABLE 3
Grouped by normal + CIN1 versus CIN2 + CIN3

| | Min. | 1st Qu. | Median | Mean | 3rd Qu. | Max. |
|---|---|---|---|---|---|---|
| Normal_CIN1 | 6.05 | 71.68 | 136.8 | 225.8 | 247.5 | 3057 |
| CIN2_CIN3 | 2.25 | 137 | 246.5 | 488 | 546.3 | 3525 |

Kruskal-Wallis rank sum test p = 7.941e−06
ANOVA p = 0.00103

TABLE 4
Grouped by normal, CIN1, CIN2, and CIN3

| | Min. | 1st Qu. | Median | Mean | 3rd Qu. | Max. |
|---|---|---|---|---|---|---|
| Normal | 6.05 | 62.79 | 99 | 215.9 | 179.4 | 3057 |
| CIN1 | 38.83 | 93.09 | 182.8 | 243.7 | 299.4 | 829.4 |
| CIN2 | 36.05 | 172 | 248.4 | 502.1 | 546.3 | 2523 |
| CIN3 | 2.25 | 93.31 | 230 | 473.7 | 524.4 | 3525 |

Kruskal-Wallis rank sum test p = 8.675e−06
ANOVA p = 0.0126

### CCL2 (MCP1, pg Protein Per 200 μg Protein)

TABLE 5
Grouped by normal + CIN1 versus CIN2 + CIN3

| | Min. | 1st Qu. | Median | Mean | 3rd Qu. | Max. |
|---|---|---|---|---|---|---|
| Normal_CIN1 | 1.2 | 4.672 | 7.015 | 14.51 | 12.25 | 184.6 |
| CIN2_CIN3 | 0 | 7.2 | 12.93 | 30.15 | 36.42 | 346.8 |

Kruskal-Wallis rank sum test p = 1.385e−05
ANOVA p = 0.00469

TABLE 6
Grouped by normal, CIN1, CIN2, and CIN3

| | Min. | 1st Qu. | Median | Mean | 3rd Qu. | Max. |
|---|---|---|---|---|---|---|
| Normal | 1.2 | 4.48 | 5.68 | 11.52 | 9.34 | 121.5 |
| CIN1 | 1.96 | 5.61 | 10.45 | 19.99 | 18.32 | 184.6 |
| CIN2 | 2.95 | 5.97 | 11.36 | 32.14 | 41.15 | 346.8 |
| CIN3 | 0 | 7.532 | 16.7 | 28.13 | 32 | 127.8 |

Kruskal-Wallis rank sum test p = 1.113e−05
ANOVA p = 0.0267

### CCL3 (MIP-1Alpha, pg Protein Per 200 μg Protein)

TABLE 7
Grouped by normal + CIN1 versus CIN2 + CIN3

| | Min. | 1st Qu. | Median | Mean | 3rd Qu. | Max. |
|---|---|---|---|---|---|---|
| Normal_CIN1 | 0 | 3.155 | 6.96 | 10.85 | 13.06 | 77.8 |
| CIN2_CIN3 | 0 | 6.02 | 12.07 | 22.33 | 23.58 | 231.9 |

Kruskal-Wallis rank sum test p = 0.0001189
ANOVA p = 0.0029

TABLE 8
Grouped by normal, CIN1, CIN2, and CIN3

| | Min. | 1st Qu. | Median | Mean | 3rd Qu. | Max. |
|---|---|---|---|---|---|---|
| Normal | 0 | 3.12 | 6.21 | 9.956 | 12.95 | 67.24 |
| CIN1 | 0.23 | 4.22 | 7.94 | 12.49 | 15.26 | 77.8 |
| CIN2 | 0 | 5.1 | 13.42 | 19.7 | 23.77 | 116.7 |
| CIN3 | 0 | 6.08 | 11.68 | 25 | 23.12 | 231.9 |

Kruskal-Wallis rank sum test p = 0.001437
ANOVA p = 0.017

### CCL4 (MIP-1Beta, pg Protein Per 200 μg Protein)

TABLE 9
Grouped by normal + CIN1 versus CIN2 + CIN3

| | Min. | 1st Qu. | Median | Mean | 3rd Qu. | Max. |
|---|---|---|---|---|---|---|
| Normal_CIN1 | 0.37 | 15.02 | 23.64 | 43.8 | 41.99 | 415.5 |
| CIN2_CIN3 | 0 | 18.59 | 42.32 | 74.93 | 74.33 | 1209 |

Kruskal-Wallis rank sum test p = 0.0023
ANOVA p = 0.062

TABLE 10
Grouped by normal, CIN1, CIN2, and CIN3

| | Min. | 1st Qu. | Median | Mean | 3rd Qu. | Max. |
|---|---|---|---|---|---|---|
| Normal | 0.37 | 11.94 | 22.24 | 37.56 | 33.67 | 310.1 |
| CIN1 | 4.58 | 17.3 | 38.71 | 55.21 | 54.24 | 415.5 |
| CIN2 | 0 | 15.03 | 45.62 | 55.43 | 79.2 | 284 |
| CIN3 | 0 | 20.52 | 40.8 | 94.76 | 70.38 | 1209 |

Kruskal-Wallis rank sum test p = 0.00532
ANOVA p = 0.0605

### IL-8 (pg Protein Per 200 μg Protein)

TABLE 11
Grouped by normal + CIN1 versus CIN2 + CIN3

| | Min. | 1st Qu. | Median | Mean | 3rd Qu. | Max. |
|---|---|---|---|---|---|---|
| Normal_CIN1 | 200.8 | 1059 | 1911 | 2865 | 3573 | 12000 |
| CIN2_CIN3 | 5.69 | 1519 | 2854 | 4001 | 5417 | 16210 |

Kruskal-Wallis rank sum test p = 0.003507
ANOVA p = 0.00988

TABLE 12
Grouped by normal, CIN1, CIN2, and CIN3

| | Min. | 1st Qu. | Median | Mean | 3rd Qu. | Max. |
|---|---|---|---|---|---|---|
| Normal | 200.8 | 1069 | 1961 | 2922 | 4066 | 12000 |
| CIN1 | 372.3 | 1056 | 1886 | 2761 | 3354 | 11690 |
| CIN2 | 358.7 | 1519 | 2813 | 4052 | 6460 | 16210 |
| CIN3 | 5.69 | 1554 | 3139 | 3949 | 5388 | 14740 |

Kruskal-Wallis rank sum test p = 0.03402
ANOVA p = 0.0818

### CONCLUSIONS

Our data indicate that using the Procarta panel MIP-1alpha, MIP-1beta and GRO-A as a combined biomarker panel achieves a specificity and a PPV which out-performs the all of the current market competitors (86% and 80% respectively). MIP1 beta alone delivers a specificity and PPV of 95% and 92% respectively. While the sensitivity—including for the combined panel—is currently suboptimal, we have a clear pathway to boosting this via (1) the use of alternative platforms which have been shown to detect lower levels of protein, and (2) the interrogation of real-time reverse transcription PCR strategies for the targets described.

### Example 2—Increased Sample Numbers

As a continuation of Example 1, further samples were examined to seek to further validate the earlier findings.

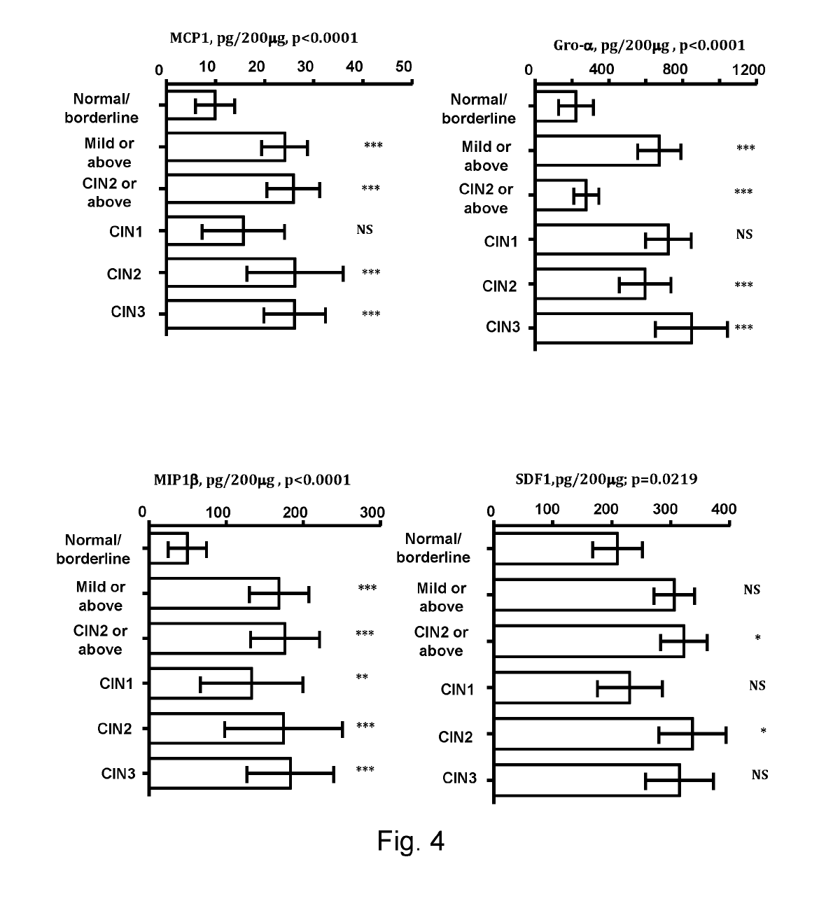

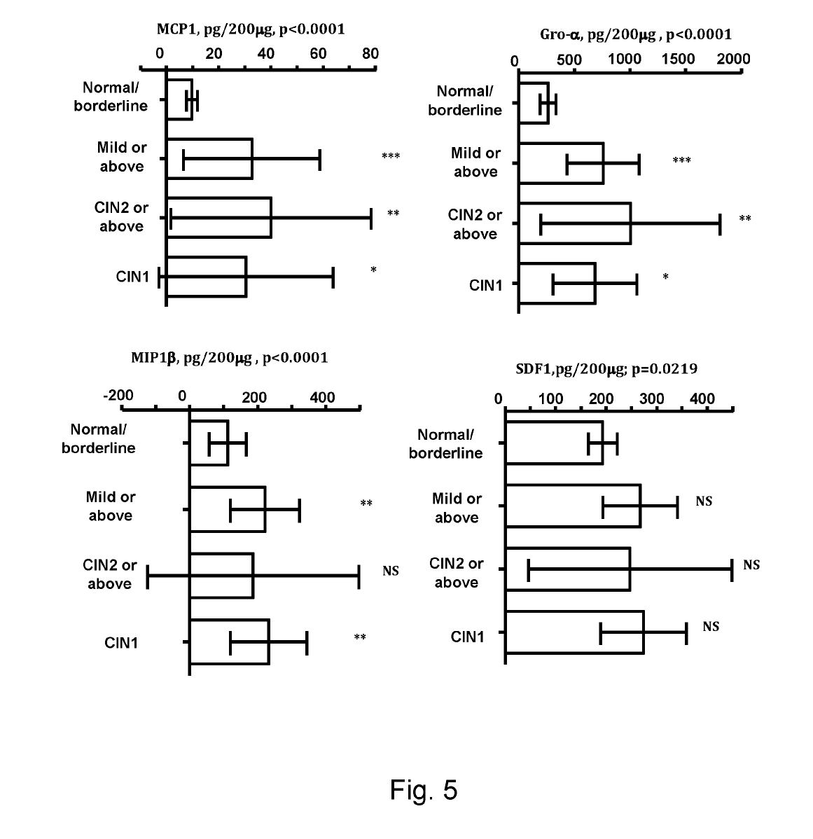

There is shown in FIGS. 4 and 5 the combined multiplex results for 482 samples. This adds in >100 samples to the earlier dataset. The results indicate that the previous conclusions are supported in these additional samples. It is observed that there is somewhat better discrimination and less noise with the HPV+ve samples, but this may just be down to the difference in numbers between the groups (326 HPV+ve and 156 HPV-ve).

The results are presented slightly differently to previously, and thus, as well as analysing samples from women with normal or borderline cytology who were not sent for colposcopy versus CIN1 2 and 3, a “sent to colposcopy” group (mild or above cytology results) and a “needed treatment at colposcopy” group (CIN2 or above) has been included.

FIG. 4 shows the results for the HPV+ve group, and additional details for this group are:

### 326 ABBOTT Test HR-HPV+Ve LBC Samples.

Multiplex Procarta kit results on extracted protein.

| | |
|---|---|
| Normal/borderline | n = 73 |
| (women not sent for colposcopy after cytology) | |
| Mild or above cytology (women sent for colposcopy) | n = 256 |
| CIN2 or above | n = 211 |
| (women who required treatment at colposcopy) | |
| CIN1 | n = 45 |
| CIN2 | n = 91 |
| CIN3 | n = 115 |

SDF1 shows smaller differences compared with the other markers. MIP1beta very clearly allows differentiation of CIN1 and above; Gro-alpha and MCP1 clearly allow differentiation of CIN2 and above.

FIG. 5 shows the results for the HPV-ve group, and additional details for this group are:

### 156 ABBOTT Test HR-HPV-Ve LBC Samples.

Multiplex Procarta kit results on extracted protein.

| | |
|---|---|
| Normal/borderline | n = 126 |
| (women not sent for colposcopy after cytology) | |
| Mild or above cytology (women sent for colposcopy) | n = 30 |
| CIN2 or above | n = 7 |
| (women who required treatment at colposcopy) | |
| CIN1 | n = 23 |

SDF1 shows no significant difference. MIP1beta allows differentiation of CIN1; Gro-alpha and MCP1 clearly allow differentiation of CIN1 and above.

### Example 3—mRNA Analysis

To further validate the approach of using mRNA expression levels as an alternative to protein levels, measurements of mRNA levels were undertaken for CCL2 and CCL5 to confirm whether differences were differences in protein levels were mirrored in differences in mRNA levels. The methodology is described in Example 1 for CCL2, and an identical method with primers and probes for CCL5 was used (Cat. No. 4331182).

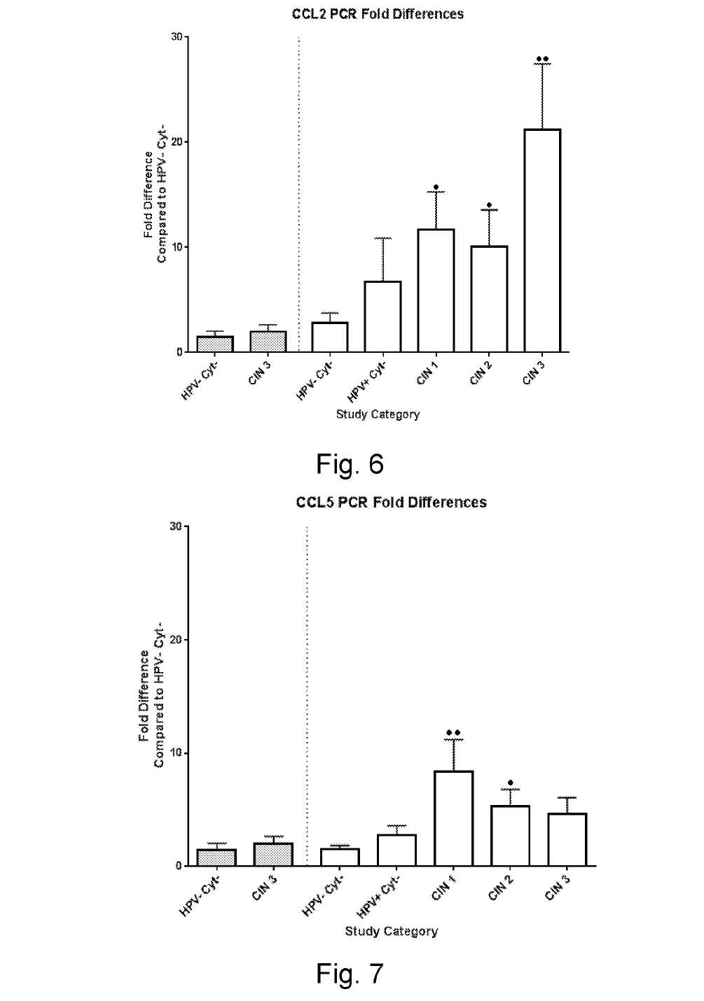

The results for CCL2 are shown in FIG. 6. Significant differences were observed between all three cytology+ groups and HPV− cytology−.

Sample numbers were as follows:
HPV− cytology− n=22,
HPV+ cytology− n=20,
CIN1 n=15,
CIN2 n=13,
CIN3 n=15

The results for CCL5 are shown in FIG. 7. Significant differences between CIN1 and CIN 2 and HPV− cytology−.

Sample numbers were as follows:
HPV− cytology− n=20,
HPV+ cytology− n=19,
CIN1 n=15,
CIN2 n=13,
CIN3 n=15

As expected, these results further support supposition that both protein levels and mRNA levels can be successfully used in the present invention.

### Example 4—Illustrative Screening Method and Preliminary Thresholding

Under the current system of cytology screening, approximately 3% of women are offered a colposcopy appointment for detected abnormality. 75% of women sent for colposcopy do not require treatment (i.e. are judged to have normal cervical morphology or CIN1). HPV testing is estimated to show overall positivity of around 10% of women. Approximately 70% of CIN1 lesions will spontaneously regress but 30% will progress. At present there is no way of distinguishing these and women with CIN1 are offered rescreening and colposcopy if necessary after 6-12 months.

Preliminary thresholding of the current dataset has been carried out using an arbitrary threshold as the median value of all HPV positive samples (n=320) for each of five chemokines (CXCI1, CXCL12, CCL2, CCL3, & CCL4). This threshold was then applied to the data from those samples which were below the treatment threshold. The data was analysed to discriminate between samples which should indicate women be sent for checking (i.e. which showed an increase in 3 or more chemokines at this threshold, indicating a higher level of tissue involvement), and those which should not need colposcopic examination (i.e. which showed an increase in 0, 1 or 2 chemokines, indicating low tissue involvement). The analysis indicated that an assay using this thresholding approach, using multiplex protein detection, would have sent only 38% (16/47) of those women found to have normal morphology or CIN1 on examination for colposcopy, i.e. representing a significant reduction in the number of women undergoing unnecessary colposcopy.

It should be noted that this screening method, and the thresholding system applied, is merely a preliminary approach, and is not intended to represent an final, optimal screen for any particular patient group or situation. However, it does serve to illustrate the utility of the present method. Optimisation of a screening method of the present invention for use in particular situations, or with particular patient groups, can be readily carried out by the skilled person.

### Discussion of Health Economics

From the 2011 census, there are 28.5 m women in the UK. Using the anticipated value of 10-15% women being HPV positive, and assuming all women are screened once every three years, approximately 950,000 to 1.4 m women would screen positive for HPV annually. This is potentially a massive burden on the NHS in terms of further investigations.

Adding an additional triage test for positive HPV screens in this patient cohort, would add an additional cost initially, but result in large savings further down the line. The benefits of this triage step are multiple. As is often the case in healthcare, the costs and savings generated by this test occur across different organisations and budget lines, so a holistic overview of the impact is required.

The main area of budget saving associated with the introduction of the triage test is reduced numbers of referrals to colposcopy. It is estimated that unnecessary colposcopy referrals may be reduced by up to 70% from their current rate. Further investigation of the numbers of women currently referred for colposcopy who have an outcome of no biopsy and no treatment is also relevant here—if the adoption of a triage test leads to a decrease in this outcome, not only is the test reducing the total numbers overall, it is also helping to improve the intrinsic performance of colposcopy as a test, which would be good for patients. Further detailed modelling work is required to pick up all the nuances of cost and clinical benefit associated with reduced demand for colposcopy, including the possible impact on waiting times etc., which is not possible within the scope of this early assessment. However a simplistic estimate of the impact can be made, as follows.

Colposcopy in England is remunerated at the outpatient appointment rate for gynaecology, £131 in 2014/15. In England, 3.57 million screening samples were examined by pathology laboratories in 2012-13. Data from the English cervical screening service suggests that 167,394 referrals to colposcopy were made from screening in 2012-13, so 4.7% of the screening samples resulted in a referral to colposcopy. So, using current prices and last year's activity (the most recent available) we can estimate the annual cost of screening-related colposcopy activity to be approximately £19.8 m for England, or £25 m for the whole of the UK (recognising that there is no comparable tariff system in some of the other countries so this is harder to assess).

Reducing these referrals by 70% would therefore save 105,000 colposcopies and £13.8 m from the current annual spend. These “savings” would occur at hospital level, in the form of reduced income. Hospitals may disinvest to cope with smaller volumes, or may reallocate their resources to the higher number of patients actually requiring treatment, rather than just colposcopy, which will result from the higher number of patients found through screening who require treatment for pre-cancerous cell changes. Hospitals in England are paid separately for this activity, so that income stream would be expected to increase, compensating in part for the loss of pure colposcopy income.

This analysis does not take into account harder to measure costs, such as patient's time, travel, childcare and intangible but significant factors such as anxiety. Patients may also consult their GP more frequently if they are called for colposcopy, which would be reduced by reducing the numbers required to take the test.

Pure savings are not however the whole story. It is also important to compare the costs of using a triage test according to the present invention (referred to as CINck) to the costs of alternative triage. If CINck is not used as a triage test, the recommended pathway would be to subject HPV positive screens to subsequent cytology screening as a triage step. Using a quoted cost of £21.36 as the lab-based cost of a liquid based cytology screen, and applying the same assumptions as above, it can be seen that the costs of this alternative triage will be £30.4 m to £20.2 m per year. While this is potentially cheaper than triage with CINck, it will not deliver the same sensitivity, meaning that some screens are interpreted as false negatives at a potentially high cost to the patients concerned. The element of subjectivity inherent in cytology based screening also remains.

On the basis of the initial estimates above, using CINck to triage HPV positive samples appears to be more expensive than cytology-based triage, but confers greater clinical advantage. The possible savings resulting from fewer colonoscopies appear to be adequate to fund this additional cost differential between the two triage approaches.

Overall, a triage test according to the present invention has the power to prevent a clinically unnecessary invasive diagnostic procedure for many women, which can be the cause of significant anxiety as well as cost to the NHS.

### REFERENCES

1. Szarewski A, Mesher D, Cadman L, Austin J, Ashdown-Barr L, Ho L, Terry G, Liddle S, Young M, Stoler M, McCarthy J, Wright C, Bergeron C, Soutter W P, Lyons D, Cuzick J (2012) Comparison of seven tests for high-grade cervical intraepithelial neoplasia in women with abnormal smears: the Predictors 2 study. J Clin Microbiol 50: 1867-1873. JCM.00181-12 [pii]; 10.1128/JCM.00181-12 [doi].
2. Dijkstra M G, van N D, Rijkaart D C, van Kemenade F J, Heideman D A, Snijders P J, Meijer C J, Berkhof J (2014) Primary hrHPV DNA testing in cervical cancer screening: how to manage screen-positive women? A POBASCAM trial substudy. Cancer Epidemiol Biomarkers Prev 23: 55-63. 1055-9965.EPI-13-0173 [pii]; 10.1158/1055-9965.EPI-13-0173 [doi].
3. Kavanagh K, Pollock K G, Potts A, Love J, Cuschieri K, Cubie H, Robertson C, Donaghy M (2014) Introduction and sustained high coverage of the HPV bivalent vaccine leads to a reduction in prevalence of HPV 16/18 and closely related HPV types. Br J Cancer 110: 2804-2811. bjc2014198 [pii]; 10.1038/bjc.2014.198 [doi].
4. De Strooper L M, van Z M, Steenbergen R D, Bleeker M C, Hesselink A T, Wisman G B, Snijders P J, Heideman D A, Meijer C J (2014) CADM1, MAL and miR124-2 methylation analysis in cervical scrapes to detect cervical and endometrial cancer. J Clin Pathol 67: 1067-1071. jclinpath-2014-202616 [pii]; 10.1136/jclinpath-2014-202616 [doi].
5. Canham M, Charsou C, Stewart J, Moncur S, Hoodless L, Bhatia R, Cong D, Cubie H, Busby-Earle C, Williams A, McLoughlin V, Campbell J D, Cuschieri K, Howie S (2014) Increased Cycling Cell Numbers and Stem Cell Associated Proteins as Potential Biomarkers for High Grade Human Papillomavirus+ve Pre-Neoplastic Cervical Disease. PLoS One 9: e115379. 10.1371/journal.pone.0115379 [doi]; PONE-D-14-30656 [pii].

1. A method of determining the clinical significance of an HPV infection in a subject, the method comprising:
providing a biological sample, preferably a tissue sample, from said subject;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection.

2. The method of claim 1 wherein subject is a human.

3. The method of claim 1 wherein the biological sample is a cervical tissue sample that comprises cervical cells.

4. The method of claim 1 wherein the HPV infection is an hrHPV infection.

5. The method of claim 1 which is a method of detecting or predicting the occurrence of HPV-induced high-grade precancerous lesions and/or HPV-induced invasive cancers.

6. The method of claim 1 which is a diagnostic method for differentiating between subjects who have an HPV infection which is likely to resolve without treatment or which does not currently merit further investigation and/or treatment from those in which a more active management is required.

7. The method of claim 1 which is for differentiating between patients likely to exhibit normal cervical tissue or CIN1 histology/low grade squamous intraepithelial lesion (LSIL) cytology, and those likely to have CIN2 or CIN3 histology/high grade squamous intraepithelial lesion (HSIL) cytology.

8. The method of claim 1 which is part of a diagnostic method performed by a clinician to allow them to decide upon a suitable course of treatment.

9. The method of claim 1 which comprises identifying an alteration in expression levels of at least one biomarker selected from the group consisting of CCL2, CCL3. CCL4, CXCL1 and CXCL12.

10. The method of claim 9 wherein the alteration is relative to:
a pre-determined reference value;
a value derived from contemporaneously obtained tissue form the subject which is not infected with HPV; or
a value derived from previously obtained sample.

11. The method of claim 9 wherein the alteration is an increase in abundance of the biomarker protein itself, or in mRNA encoding the biomarker protein.

12. The method of claim 1 comprising the step of obtaining a sample of cervical tissue from a subject.

13. The method of claim 1 which is a method of screening a subject for risk of developing cervical cancer, the method comprising:
obtaining a cervical tissue sample containing a plurality of cervical cells from the subject;
detecting the presence of HPV infected cells in the sample;
determining the abundance, in at least a portion of said cervical sample, of at least one biomarker, or the abundance of mRNA encoding for said at least one biomarker, the at least one biomarker selected from the group consisting of CCL2, CCL3, CCL4, CXCL1 and CXCL12; and
determining therefrom an indication of risk of the subject developing cervical cancer.

14. The method of claim 1 comprising the step of isolating protein from cells in the sample.

15. The method of claim 1 comprising the step of isolating nucleic acids, especially mRNA, from cells in the sample.

16. The method of claim 1 comprising the step of selecting subjects to undergo further investigation and/or selecting subjects for treatment.

17. The method of claim 1 comprising determining the abundance of at least two of CCL2, CCL3, CCL4, CXCL1 and CXCL12.

18. (canceled)

19. The method of claim 1 comprising determining the abundance of at least three of CCL2, CCL3, CCL4, CXCL1 and CXCL12.

20. (canceled)

21. The method of claim 1 comprising determining the abundance of at least four of CCL2, CCL3, CCL4, CXCL1 and CXCL12.

22. The method of claim 1 comprising determining the abundance of at least CCL2, CXCL1 and CXCL12 and/or the abundance of mRNA encoding CCL2, CXCL1 and CXCL12.

23. The method of claim 1 comprising determining the abundance of CCL2, CCL3, CXCL1 and CXCL12, and/or the abundance of mRNA encoding CCL2, CCL3, CXCL1 and CXCL12.

24. The method of claim 1 comprising determining the abundance of at least CCL3, CCL4 and CXCL1, and/or the abundance of mRNA encoding CCL3, CCL4 and CXCL1.

25. (canceled)

26. The method of claim 1 further comprising determining of the abundance of CCL28, Chemerin, CXCL5, IL-16, CXCL11, CXCL9, CCL5, CCL17, GP130 and/or Transferrin Receptor, and/or the abundance of mRNA encoding CCL28, Chemerin, CXCL5, IL-16, CXCL11, CXCL9, CCL5, CCL17, GP130 and/or Transferrin Receptor.

27. The method of claim 1 comprising determining the abundance of one or more reference biomarkers, such as IL-8, or total protein level or mRNA level.

28. The method of claim 1 comprising testing for the presence of non-HPV infections.

29. (canceled)

30. A method of treatment of a subject, the method comprising the steps of:
providing a biological sample from said subject;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection;
deciding whether treatment is required; and
if treatment is required, deciding upon an appropriate treatment and carrying out said treatment.

31. A method for monitoring the efficacy of a treatment for HPV infection or a lesion associated with an HPV infection in a subject, the method comprising:
providing a suitable biological sample, preferably a tissue sample, from said subject which is undergoing or which has undergone treatment;
determining the abundance in at least a portion of said sample of at least one biomarker, or the abundance of mRNA encoding said at least one biomarker, the at least one biomarker selected from the group consisting of CCL2, CCL3, CCL4, CXCL1 and CXCL12;
determining therefrom the clinical significance of the HPV infection; and
based upon the result, assessing the efficacy of the treatment.

32. The method of claim 31 which comprises comparing the results obtained with the results obtained in an equivalent procedure carried out previously.

33. (canceled)

34. (canceled)
